(19) United States
(12) Patent Application Publication
(10) Pub. No.: US 2017/0325757 A1
(43) Pub. Date: Nov. 16, 2017

(54) PATIENT SUPPORT APPARATUS WITH A COUPLING UNIT AS WELL AS A MEDICAL IMAGING APPARATUS WITH A COUPLING UNIT

(71) Applicant: Siemens Healthcare GmbH, Erlangen (DE)

(72) Inventor: Stephan BIBER, Erlangen (DE)

(73) Assignee: Siemens Healthcare GmbH, Erlangen (DE)

(21) Appl. No.: 15/593,513

(22) Filed: May 12, 2017

(30) Foreign Application Priority Data
May 12, 2016 (DE) 102016208220.5

(51) Int. Cl. A61B 6/04; A61B 6/00; A61G 13/02; A61G 13/12

(52) U.S. Cl. CPC A61B 6/04; A61G 13/126; A61B 6/42; A61G 13/02

(57) ABSTRACT

A patient support apparatus has a movable patient couch, a support unit, and a coupling unit for coupling with a detector unit of a medical imaging apparatus, wherein the patient support apparatus has a damping unit that is designed to damp and/or compensate forces which act on the patient support apparatus coupled with the detector unit.

### BACKGROUND OF THE INVENTION

### Field of the Invention

The present invention relates to a patient support apparatus with a movable patient couch, a support unit and a coupling unit for coupling with a detector unit (data acquisition scanner) of a medical imaging apparatus. Furthermore the present invention relates to a medical imaging apparatus with a detector unit, and a patient support apparatus that can be coupled to the detector unit, wherein the detector unit has a coupling unit for coupling with the patient support apparatus.

### Description of the Prior Art

With medical imaging apparatuses, for a medical imaging examination a patient is introduced or moved into a patient receiving zone by means of a patient support apparatus. This patient receiving zone is surrounded by a detector unit of the medical imaging apparatus.

With patient support apparatuses which are not permanently connected in a fixed manner to the medical imaging apparatus, the patient support apparatus must have a coupling unit in order to couple to the medical imaging apparatus, in particular to the detector unit of the medical imaging apparatus. In the event of collisions between a further unit and the patient support apparatus, damage to the patient support apparatus may occur, which results on account of transverse forces acting on the patient support apparatus.

### SUMMARY OF THE INVENTION

An object of the present invention is to provide a damping and/or compensation of transverse forces acting on the patient support apparatus.

The invention is based on a patient support apparatus with a movable patient couch, a support unit and a coupling unit for coupling with a detector unit of a medical imaging apparatus.

The patient support apparatus has a damping unit, which is designed to damp and/or compensate forces which act on the patient support apparatus coupled with the detector unit.

Here the medical imaging apparatus can be any medical imaging apparatus that appear appropriate to those skilled in the art, such as a magnetic resonance apparatus, a computed tomography apparatus, a PET apparatus, a C-arm apparatus etc. The detector unit (scanner) is preferably designed to record medical imaging data by operation of the medical imaging apparatus. If the medical imaging apparatus is formed by a magnetic resonance apparatus for instance, then the detector unit is formed by a scanner with a basic field magnet and a radio-frequency antenna.

The patient support apparatus preferably includes a movable patient couch, by which a patient can be introduced and/or moved forward into a patient receiving zone of the detector unit. Furthermore, the patient support apparatus also has a support, which supports the patient couch, in particular a subregion of the patient couch that is not arranged within the patient receiving zone, against the direction of the force of gravity acting on the patient support apparatus.

The patient support apparatus is designed to be couplable to the detector unit of the medical imaging apparatus by the coupling unit. The patient support apparatus can have a first coupling state, in which the patient support apparatus is separated and/or decoupled from the detector unit, and a second coupling state, in which the patient support apparatus couples to the detector unit. The coupling unit preferably is a mechanical coupling unit.

The coupling unit is preferably arranged in a front region of the patient support apparatus. The front region is preferably the region of the patient support apparatus that, by an insertion movement of the patient support apparatus into a patient receiving zone of the detector unit, is first to extend into the patient receiving zone. Moreover, the detector unit can also have a coupling element that corresponds to the coupling unit of the patient support apparatus, which coupling element is preferably arranged in an insertion region of the patient receiving zone or the detector unit. The insertion region preferably comprises an opening region of the patient receiving zone, wherein the patient support apparatus, in particular the front region of the patient support apparatus, couples with the detector unit, in particular the insertion region of the patient receiving zone.

The damping unit is designed to damp forces that act on the patient support apparatus coupled with the detector unit. The damping unit absorbs and/or compensates for additional forces that do not result from the actual coupling of the patient support apparatus with the detector unit, without the position of the patient couch being affected in the process. For instance, the damping unit can absorb forces that result from a collision between a further unit and/or a user and the patient couch and/or the patient support apparatus. The damping unit has at least one damping element, which is elastically deformable in order to absorb and/or compensate for the forces.

High lateral impulses acting on the patient support apparatus can be absorbed and/or compensated for by the invention so that the coupling unit can be effectively protected from these transverse forces. Moreover, the coupling unit can also be protected from unwanted damage, wherein the damage may be caused by strong lateral impulses and/or transverse forces. The lateral impulses and/or transverse forces can result, for instance, from bumps and/or collisions between the patient support apparatus and further units and/or a user. This is particularly advantageous with patient support apparatuses that are supported exclusively by a support base and are not supported on the scanner and/or by the scanner.

In a further embodiment of the invention, the coupling unit structurally includes the damping unit. In this way a simple and robust coupling unit can be provided for coupling of the patient support apparatus with the detector unit, which can, moreover, reduce the installation effort when installing a medical imaging apparatus. Moreover, a soft and/or elastic coupling can be achieved advantageously between the patient support apparatus and the detector unit (scanner).

In a further embodiment of the invention, the damping unit is designed to at least partially compensate for forces that are aligned transversely to a weight acting on the patient support apparatus. This advantageously enables unwanted transverse forces acting on the patient support apparatus to be compensated for so that damage to and/or impairment of the patient support apparatus can be prevented. Furthermore, a position of the reclining surface, in particular a distance between a reclining surface and a floor surface, can essentially be kept constant so that an insertion process of the patient couch into the patient receiving zone of the detector unit need not be interrupted. The position of the reclining surface, in particular the distance between the reclining surface and the floor surface, can preferably also be maintained regardless of a patient's weight and need not be readjusted as a function of a patient's weight.

Alternatively or in addition the damping unit can be designed to at least partially compensate for forces which are aligned transversely to an insertion direction of the patient support apparatus into the detector unit. This likewise enables unwanted transverse forces acting on the patient support apparatus to be compensated for so that damage to and/or impairment of the patient support apparatus can be prevented.

In a further embodiment of the invention, the support includes the damping unit. In this way an advantageous compensation of forces acting on the patient support apparatus can be achieved and thus an effective protection can be provided for the coupling unit.

If the damping unit has a spring unit, a particularly simple and cost-effective damping unit can be provided. Such a spring unit includes a spring element, which is designed to compensate for and/or absorb the unwanted forces nevertheless acting on the patient support apparatus.

Furthermore the invention encompasses a medical imaging apparatus having a detector unit, and a patient support apparatus that can be coupled to the detector unit, wherein the detector unit has a coupling unit for coupling with the patient support apparatus, wherein the coupling unit has a damping unit, which is designed to damp and/or compensate for forces which act on the patient support apparatus coupled with the detector unit. In this way a simple and robust coupling unit can be provided for coupling of the patient support apparatus with the detector unit, which can moreover reduce the installation effort when installing a medical imaging apparatus. Moreover, advantageously a soft and/or elastic coupling can be achieved between the patient support apparatus and the detector unit on account of the damping unit.

In a further embodiment, the patient support apparatus has a movable patient couch, a support unit, a coupling unit for coupling with a detector unit of a medical imaging apparatus and a damping unit, wherein the damping unit is designed to damp and/or compensate for forces which act on the patient support apparatus coupled to the detector unit.

Strong lateral impulses acting on the patient support apparatus can be absorbed and/or compensated for by the invention so that the coupling unit can be effectively protected from these transverse forces. Moreover, the coupling unit can also be protected from unwanted damage, wherein the damage may be caused on account of high lateral impulses and/or transverse forces. The lateral impulses and/or transverse forces can result for instance from bumps and/or collisions between the patient support apparatus and further units and/or a user.

The advantages of the inventive medical imaging apparatus essentially correspond to the advantages of the inventive patient support apparatus, as explained in detail above.

### BRIEF DESCRIPTION OF THE DRAWINGS

FIG. 1 shows a medical imaging apparatus and a patient support apparatus in a schematic representation from above.

FIG. 2 shows the medical imaging apparatus and the patient support apparatus in a schematic side view.

### DESCRIPTION OF THE PREFERRED EMBODIMENTS

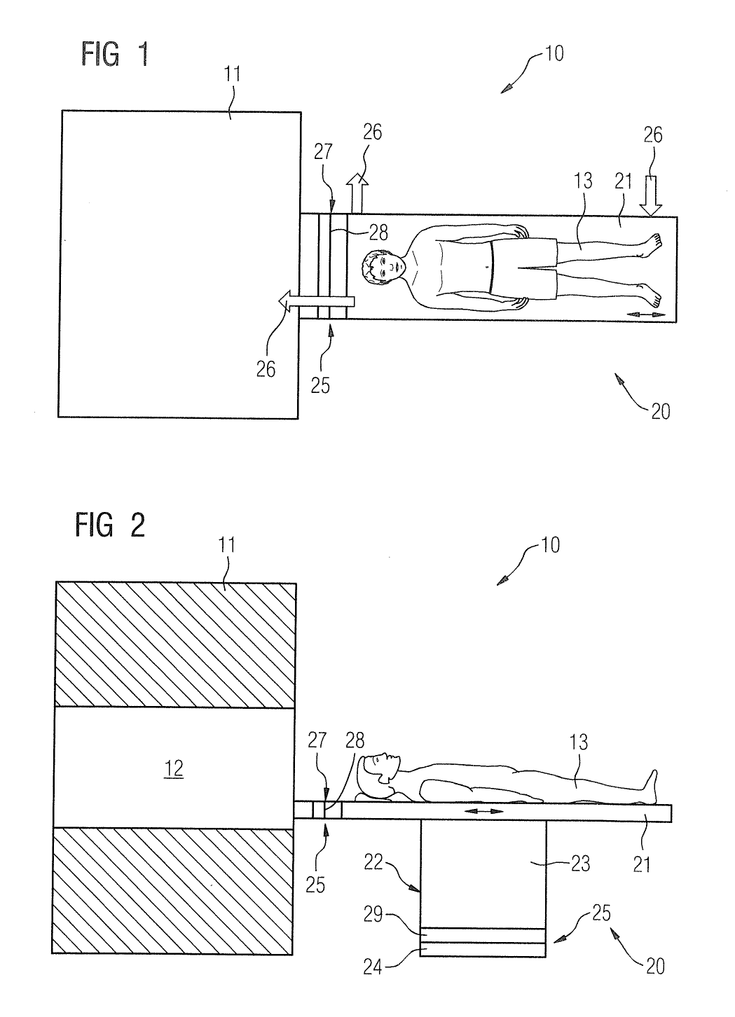

FIGS. 1 and 2 show a schematic representation of a medical imaging apparatus 10. In the present exemplary embodiment the medical imaging apparatus 10 is formed by a magnetic resonance apparatus and so, as an example, the present invention is explained on the basis of the magnetic resonance apparatus. The present invention is not restricted to the embodiment of the medical imaging apparatus 10 on a magnetic resonance apparatus and further embodiments of the medical imaging apparatus 10 are readily conceivable.

The magnetic resonance apparatus has a detector unit 11 (scanner), which includes a basic field magnet and a radio-frequency antenna and gradient coils. The detector unit 11 has a patient receiving zone 12 for receiving a patient 13. In the present exemplary embodiment the patient receiving zone 12 is embodied to be cylindrical and in a peripheral direction is surrounded in a cylindrical manner by the detector unit 11. In principle, however, an embodiment of the patient receiving zone 12 which deviates from that described is readily conceivable.

The patient 13 can be introduced into the patient receiving zone 12 by means of a patient support apparatus 20 of the medical imaging apparatus 10, in the present case the magnetic resonance apparatus. For this purpose the patient support apparatus 20 has a patient couch 21 which is configured to be movable within the patient receiving zone 12. Furthermore, the patient support apparatus 20 has a support unit 22, which is designed to support and/or hold the patient couch 21, wherein the support unit 22 only supports the subregion of the patient couch 21, which is arranged and/or positioned outside of the patient receiving zone 12. The support unit 22 has a support foot 23 and a base plate 24, wherein the support foot 23 is supported and/or mounted on a floor of an examination room by way of the base plate.

In the present exemplary embodiment, the patient support apparatus 20 is formed by a patient support apparatus 20 that can be coupled with the detector unit 11. The patient support apparatus 20 is thus not permanently connected in a fixed manner to the detector unit 11. To this end the patient support apparatus 20 has a coupling unit 25, which is formed by a mechanical coupling unit. Moreover, the medical imaging apparatus 10 also has a coupling unit 14, which is embodied so as to correspond to the coupling unit of the patient support apparatus 20.

In order to move the patient couch 21 forward into the patient receiving zone 12, the patient support apparatus 20 must firstly couple to the detector unit 11 by the coupling unit 25. The coupling unit 25 is arranged in a front region of the patient support apparatus 20, wherein the front region comprises the end region of the patient support apparatus 20 which is arranged in the direction of an insertion direction of the patient support apparatus 20 in the side of the patient support apparatus 20 facing the detector unit 11. The coupling unit 14 of the detector unit 11 is preferably arranged in an opening region of the patient receiving zone 12.

If the patient support apparatus 20 is in a state coupled to the detector unit 11, collisions between further units and/or a user and the patient support apparatus 20 may cause the patient support apparatus 20 to be displaced and thus the patient couch 21 to tilt within the patient receiving zone 12 and/or may cause damage to the coupling unit 25. This damage can result from transverse forces 26, as indicated for instance in FIG. 1.

To avoid such damage and/or for a safe design of the patient support apparatus 20, the patient support apparatus 20 has a damping unit 27, which is designed to damp and/or compensate for forces, in particular transverse forces 26, which act on the patient support apparatus 20 in a state coupled to the detector 11. Here the damping unit 27 has a spring unit with individual spring elements for damping and/or compensating for forces, in particular transverse forces 26, acting on the patient support apparatus 20 coupled to the detector unit 11.

In such a case the damping unit 27 has a first damping element 28, which is formed by a spring element, which is contained in the coupling unit 25 (FIGS. 1 and 2). Furthermore the damping unit 27 has a second damping element 29, which is formed by a spring element, which is contained in the support unit 22 (FIG. 2). In the present exemplary embodiment the second damping element 29 is arranged between the support foot 23 and the base plate 24. In principle an arrangement which deviates from that described is readily conceivable.

Furthermore the coupling unit 14 of the medical imaging apparatus 10 also has a damping unit 15 with a damping element which likewise includes a spring unit.

The damping unit 27 of the patient support apparatus, in particular the first damping element 28 and the second damping element 29, and the damping unit 14, in particular the damping element, of the medical imaging apparatus 10 are designed to damp and/or compensate for transverse forces 26 acting on the patient support apparatus 20. Here the damping unit 27 of the patient support apparatus, in particular the first damping element 28 and the second damping element 29, and the damping unit 14, in particular the damping element, of the medical imaging apparatus 10 are designed to at least partially damp and/or compensate for forces, in particular transverse forces 26, which are aligned transversely to a weight acting on the patient support apparatus 20 and transversely to the insertion direction of the patient support apparatus 20 into the patient receiving zone 12 of the detector unit 11. These transverse forces 26 can thus no longer act on the coupling of the patient support apparatus 20 with the detector unit 11 so that a patient's safety 13 is advantageously increased.

In an alternative embodiment of the invention, the damping unit 27 can have only the first damping element 28 or only the second damping element 29. Moreover, in an alternative embodiment of the invention, only the damping unit 27 of the patient support apparatus 20 or only the damping unit 15 of the medical imaging apparatus 10 can be implemented and/or present. Furthermore, an embodiment of the damping unit 27 which deviates from a spring unit is also possible.

Although modifications and changes may be suggested by those skilled in the art, it is the intention of the Applicant to embody within the patent warranted hereon all changes and modifications as reasonably and properly come within the scope of the Applicant's contribution to the art.

1. A patient support apparatus for use with a detector unit of a medical imaging apparatus, said patient support apparatus comprising:
a movable patient couch, a support unit, and a coupling unit for coupling with the detector unit; and
a damping unit configured to damp and/or compensate forces that act on the patient support apparatus coupled with the detector unit.

2. The patient support apparatus as claimed in claim 1, wherein the coupling unit comprises the damping unit.

3. The patient support apparatus as claimed in claim 1, wherein the damping unit is configured to at least partially compensate for forces that are aligned transversely to a weight acting on the patient support apparatus.

4. The patient support apparatus as claimed in claim 1, wherein the damping unit is configured to at least partially compensate for forces that are aligned transversely to an insertion direction of the patient support apparatus into the detector unit.

5. The patient support apparatus as claimed in claim 1, wherein the support unit comprises the damping unit.

6. The patient support apparatus as claimed in claim 1, wherein the damping unit comprises a spring unit.

7. A medical imaging apparatus comprising:
a detector unit;
a patient support apparatus that can be coupled to the detector unit; and
the detector unit comprising a coupling unit for coupling with the patient support apparatus, said coupling unit comprising a damping unit configured to damp and/or compensate forces that act on the patient support apparatus coupled to the detector unit.
